(19) United States
(12) Patent Application Publication
(10) Pub. No.: US 2003/0185785 A1
(43) Pub. Date: Oct. 2, 2003

(54) Composition for hair straightening and method for its preparation

(76) Inventor: Daniel Moyal, Tel Aviv (IL)

Correspondence Address: DANIEL MOYAL, PO BOX 6791, TEL AVIV, IL

(21) Appl. No.: 10/108,496

(22) Filed: Mar. 29, 2002

(51) Int. Cl. A61K 35/78; A61K 7/06

(52) U.S. Cl. 424/74; 424/777

(57) ABSTRACT

A composition for hair treatment comprising a mixture of gallnuts and copper oxide, each in powder form, in the ratio of about 75 gram copper oxide for each 1 kg of gallnuts, and water. It may further include one or more of the following components: cooking or olive oil and/or clove and/or almond shells and/or pomegranate shells and/or common salt and/or soap or wash paste and/or banana shells. A method for preparing a composition for hair treatment, comprising the steps: A) Taking 1 kg. gall-nuts and dividing them up into 2 equal parts. B) Mixing 150 grams of cloves with 75 grams of copper oxide C) Grinding up everything into powder D) Preparing an alcoholic liquid; E) Mixing with the alcoholic liquid.

### CROSS-REFERENCE TO RELATED APPLICATIONS

The present application is related to the application No. 119247 filed on Sep. 12, 1996 in Israel, now abandoned, and to an application filed in Israel on March 2002.

### STATEMENT AS TO RIGHTS TO INVENTIONS MADE UNDER FEDERALLY SPONSORED R&D

There was no Federal sponsoring for the present invention, therefore there are no rights deriving therefrom.

### FIELD OF THE INVENTION

This invention relates to hair treatment compositions, and more particularly to such compositions for hair straightening and dyeing.

### BACKGROUND OF THE INVENTION

Heretofore, various compositions were used for hair straightening, mainly based on chemical preparations with acid or alkalic properties. These chemicals have strong odors, are active for only a short period of time, and usually the preparations are not sold in a ready-to-use state, but need further preparations by the coiffeur.

For example, an acid composition for shaping hair—comprising carboxylic acid, sulphonic acid and organic solvent.

Another composition for correcting frizzy hair without damage to hair—comprises keratin-reducing substances e.g. thioglycolic acid or N-acyl cysteine(s), one or a mixt. of mono-, di- or tri-hydric alcohol(s) and N-alkyl pyrrolidone(s) and water.

Other means for hair straightening include mechanical and/or thermal means.

It is desirable to add softness to the hair.

Curly hair is generally dry. It may be desirable to reduce or eliminate the dryness of the hair.

Yet another problem is the split ends of the hair.

Prior art methods apparently do not solve the above problems.

For example, Carter U.S. Pat. No. 4,242,567 details an electrically heated hair straightener with PTC heater pellets, which has scissor-like clamp housings pivoted on handle and heater pellets aligned on plate.

Another curly hair straightening method involves processing curly hair between iron globe and iron plate.

ZABOUB FR 2672491 describes a hair straightening and dyeing composition comprising a mixture of oak-gall, olive oil, cloves and copper oxide used in conjunction with artichoke leaves. This composition is different from that detailed in the present invention; moreover, the patent does not include a description of the method to be used for its preparation.

ZABOUB FR 2692482 describes a composition for treating the hair, containing ground gall-nuts cooked in olive oil, ground cloves, powdered Cu oxide, artichoke juice, and opt. NaCl, clay and neutral or hennaed razzoul. Composition used for dyeing and strengthening the hair.

It may also contain ground gall-nuts cooked in olive oil, followed by addition of ground cloves and powdered Cu oxide. The mixture is put into hot water, preferably contaning salt, to which artichoke juice has been added, and is held at a low temperature until it become pasty.

It is an objective of the present invention to provide for a hair straightening and dyeing composition with means for overcoming the abovedetailed deficiencies, and a method for its preparation.

### SUMMARY OF THE INVENTION

According to the present invention, there is provided a hair straightening and dyeing composition without undesired odors, having a long lasting effect and based mainly on natural, vegetable components.

Moreover, the composition adds softness to the hair.

Curly hair is generally dry. The additions described above, either reduce or eliminate the dryness of the hair.

Furthermore, the new composition helps to prevent split ends of the hair.

According to one aspect of the present invention, the composition comprises a mixture of gallnuts, copper oxide, clove, almond shells, pomegranate shells, common salt (crystalline), soap or wash lotion, dried banana shells and cooking or olive oil, each in a predefined percentage quantity.

According to a second aspect of the present invention, there is detailed a method of preparing the composition.

According to a third aspect of the present invention, the addition of vinegar and arrack results in a composition which, in addition to its hair straightening properties, also has the effect of dyeing white hair to black.

Further objects, advantages and other features of the present invention will become obvious to those skilled in the art upon reading the disclosure set forth hereinafter.

### BRIEF DESCRIPTION OF THE DRAWINGS

FIG. 1 illustrates a Method 1 for preparing a hair treatment composition.

FIG. 2 illustrates a Method 2 of use for the hair treatment composition.

FIG. 3 illustrates a Method 3 for preparing a minimal composition.

FIG. 4 illustrates another Method 4 for preparing a composition for hair treatment.

FIG. 5 illustrates yet another Method 5 for preparing a composition for hair treatment.

### DETAILED DESCRIPTION OF THE PREFERRED EMBODIMENTS

A preferred embodiment of the present invention will now be described by way of example and with reference to the accompanying drawings.

Note 1: Although specific quantities may be specifed in the following Methods and Compositions, it is to be understood that other quantites may be used as well, provided the relative proportions are preserved. That is, the same percentage of each component is used.

Note 2: Where specific quantities are indicated, it is to be understood that the ratio of the components may be changed to some extent (for example about ±15%) while still performing the desired functions.

The above Notes allow for variations in the presented Methods and Compositions, without departing from the scope of the present invention.

Method 1

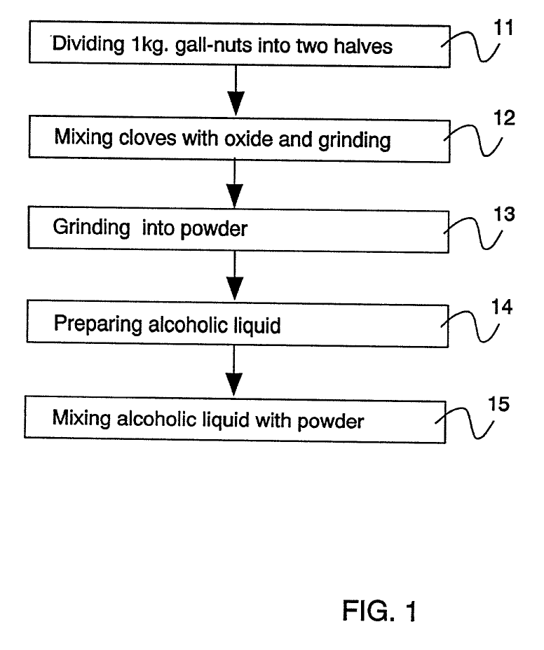

A method (see FIG. 1) for preparing a hair treatment composition comprises the following steps:

A) Taking 1 kg. gall-nuts and dividing them up into 2 equal parts, a first part and a second part.

Roasting with salt the first part for about 1 to 4 hours.

Frying in oil the second part for about 15 minutes [11];

B) Mixing 150 grams of cloves with 75 grams of copper oxide and grinding it up into powder.

It is advisable to heat the cloves a little, for about 1 minute, before grinding them up [12];

C) Grinding up everything into powder and mixing [13];

D) Preparing an alcoholic liquid, containing about 20% to 50% alcohol [14];

E) Mixing the alcoholic liquid which has been prepared in Step D, with the powder which has been prepared in Step C;

Mixing the above components initiates a chemical reaction, to create a compound with the stated properties [15].

The above method can be used for preparing a compositions for hair straightening and dyeing.

Optional Additions to the Above Compositions

Add each of the following components or a combination thereof:

a. 5 grams of dry pomegranate peels.

b. 3 grams of dry banana peels.

c. 1.5 grams of dry artichoke leaves.

d. 2 grams of dry almond shells.

e. 5 grams of olive pits.

Grinding up these components and adding them to the powder obtained in step (C).

These components add softness to the hair.

Curly hair is generally dry. The additions described above, either reduce or eliminate the dryness of the hair.

Furthermore, the new composition helps to prevent split ends of the hair.

Method 2

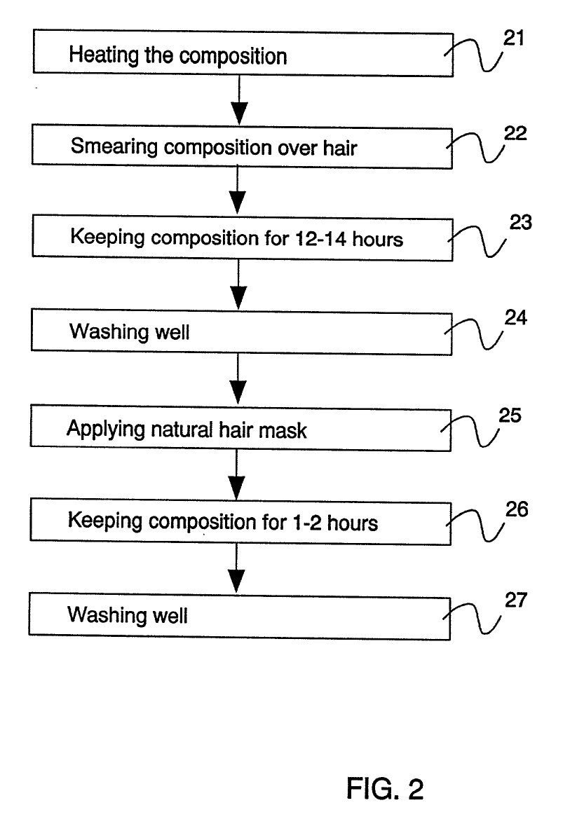

A method of use (see FIG. 2) for the hair treatment composition, comprises the following steps:

A) Heating the composition until it is warm (close to body temperature, that is about 20° C. to 30° C.) [21];

B) Smearing the composition over the hair, using the same method as with presently used materials [22];

C) Keeping the composition for about 12 to 14 hours [23];

D) Washing well with water and shampoo [24];

E) Applying a natural hair mask made of olive oil with an egg yolk [25];

F) Keeping the composition for about 1 to 2 hours [26];

G) Washing well with water and shampoo [27].

It is recommended that the person using the composition should be relaxed and/or sleeping during the 12 hours treatment, for a more effective result.

In case of very dry hair, a quantity of 10% pure olive oil may be added to the composition.

A composition for hair straightening may be prepared from the following components, each in a preferred quantity as detailed:

a. gallnuts—1 kg

b. copper oxide—75 gram

c. water—600 gram

d. cooking or olive oil—variable quantity

e. clove—50 to 100 gram

f. almond shells (optional)—50 gram

g. pomegranate shells—50 to 100 gram

h. common salt (crystalline) 1 kg

i. soap or wash paste—variable quantity

j. banana shells—50 to 100 gram

k. vinegar and/or arrack—about 8 teaspoons to 1 kg composition.

Notes:

1. The abovedetailed composition can be used with the methods illustrated in the present disclosure.

2. It is important to keep the relative quantity of the copper oxide (b), at about the ratio of 75 gram for each kilogram of gallnuts (a); a larger quantity may damage the hair, and a smaller quantity may not have the desired hair straightening effect. The quantity of the other ingredients may vary.

3. Improved performance is achieved with the addition of one or more of the following components: cooking or olive oil and/or clove and/or almond shells and/or pomegranate shells and/or common salt (crystalline) and/or soap or wash paste and/or banana shells.

4. Various quantities of the composition may be prepared, while keeping about the same relative proportions of each of its components. The ratio of the components may be changed to some extent (for example about ±15%) while still performing the desired functions.

5. Almond shells (about 50 gram per each 1 kg composition) add a sheen to hair, and are an optional component.

6. Various embodiments of the present invention may be implemented.

For example, whereas the preferred embodiment uses all the abovedetailed components, only part of the components (d, e, f, g, h, i, j, k) detailed above may be used. The composition should include, however, components (a, b) as detailed above, as well as water (c).

The minimal composition for hair treatment thus comprises only a mixture of gallnuts and copper oxide, each in powder form, in the ratio of about 75 gram copper oxide for each 1 kg of gallnuts, and water.

Method 3

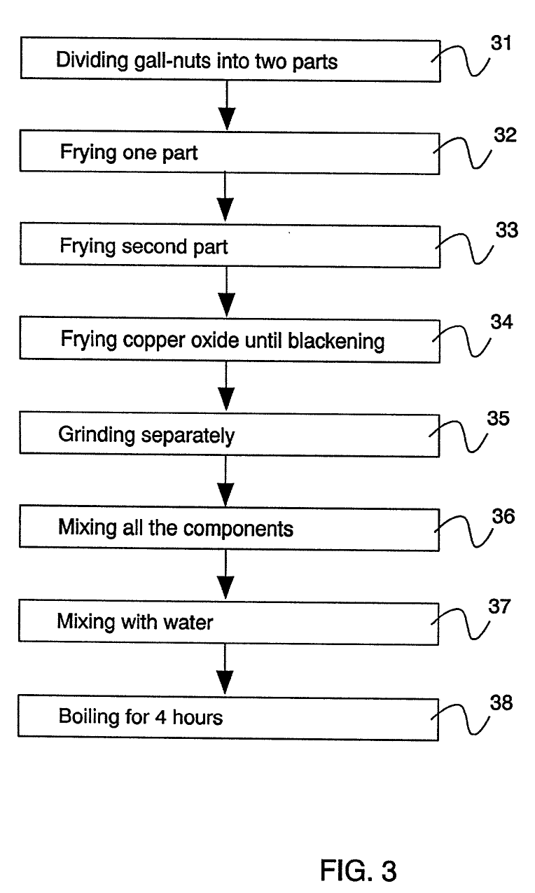

A method (see FIG. 3) for preparing a minimal composition comprises the following steps:

A) Dividing the gallnuts (a) into two halves, 0.5 kg each [31];

B) Frying half the gallnuts for about an hour, until the gallnuts “explode”, that is the shells open [32];

C) Frying the other half of the gallnuts in deep oil for about 20 to 30 minutes, until the gallnuts become soft [33];

D) Frying the copper oxide (b) until blackening in a frying pan.

The process takes about 10 to 15 minutes, and the copper oxide should be mixed continuously [34];

E) Grinding separately each one of the above resulting components [35];

F) Mixing well all the components together [36];

G) Mixing the resulting powder or paste with water (C), in the ratio of 1 kg powder with 600 gram water; alternately, mix about 10 cups of the composition with about 5 to 6 cups of water [37];

H) Heating until boiling temperature and keeping it boiling for about 4 hours [38].

Method 4

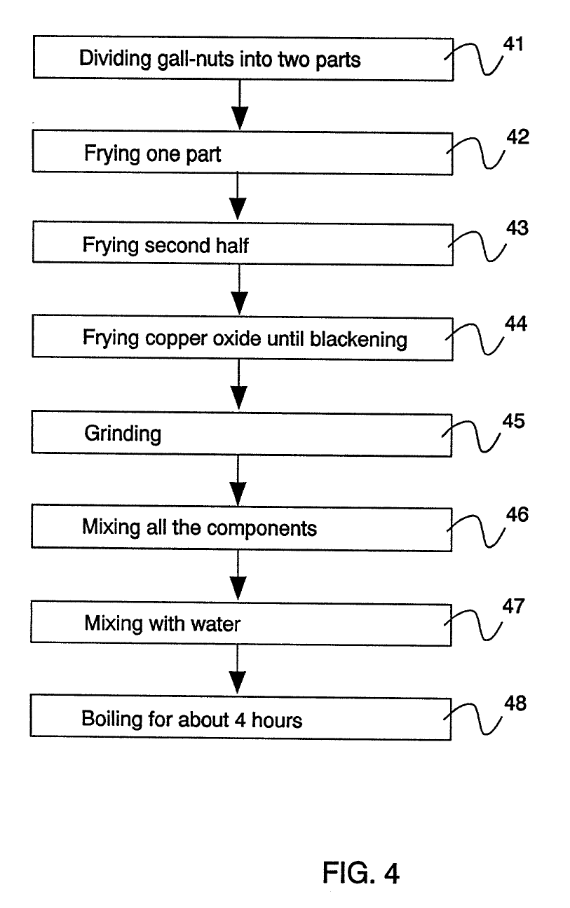

Another method (see FIG. 4) for preparing a composition for hair treatment, comprises the following steps:

A) Dividing the gallnuts into two halves, 0.5 kg each [41];

B) Frying half the gallnuts for about an hour, until the gallnuts “explode”, that is the shells open [42];

C) Frying the other half of the gallnuts in deep oil for about 20 to 30 minutes, until the gallnuts become soft [43];

D) Frying the copper oxide until blackening in a frying pan. The process takes about 10 to 15 minutes, and the copper oxide should be mixed continuously [44];

E) Grinding separately each one of the above resulting components [45];

F) Mixing well all the components together [46];

G) Mixing the resulting powder or paste with water, in the ratio of 1 kg powder with 600 gram water; alternately, mix about 10 cups of the composition with about 5 to 6 cups of water [47];

H) Heating until boiling temperature and keeping it boiling for about 4 hours [48].

Method 5

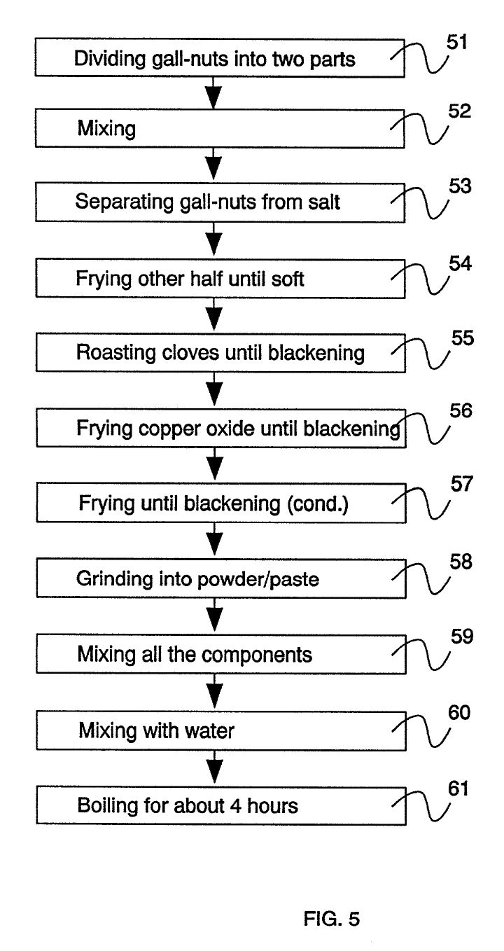

Yet another method (see FIG. 5) for preparing a composition for hair treatment, comprises the following steps:

A) Dividing the gallnuts into two halves, 0.5 kg each [51];

B) Mixing half the gallnuts with the wash paste; adding the salt and frying for about an hour, until the gallnuts “explode”, that is the shells open [52];

C) Separating the gallnuts from the salt in the mixture resulting from (B) above [53];

D) Frying the other half of the gallnuts in deep oil for about 20 to 30 minutes, until the gallnuts become soft [54];

E) Roasting the cloves in a frying pan until blackening, then removing from fire. The process takes about ten minutes, and the cloves should be mixed continuously [55];

F) Frying the copper oxide until blackening in a frying pan.

The process takes about 10 to 15 minutes, and the copper oxide should be mixed continuously [56];

G) If the almond shells and the pomegranate shells are fresh, then they should be fried until blackening; if they are dried, then this stage is to be skipped [57];

H) Grinding separately each one of the above resulting components: the gallnuts, cloves, copper oxide; grinding into a powder or paste state [58];

I) Mixing well all the components together [59];

J) Mixing the resulting powder or paste with water, in the ratio of about 1 kg powder with 600 gram water; alternately, mixing about 10 cups of the composition with about 5 to 6 cups of water [60];

K) Heating until boiling temperature and keeping it boiling for about 4 hours [61];

L) Optional (if the composition is to be used for dyeing white hair): add vinegar and/or arrack (K) to the composition.

Preferred quantity: about two teaspoons of liquid (k) to 250 gram composition [62].

In addition to the abovedetailed uses for hair straightening and dyeing, the composition has other uses as well. For example, the composition can be used for correcting curly hair, for hair curling, as well as for treating dry hair or hair with split ends.

Moreover, the composition adds softness to the hair.

Curly hair is generally dry. The additions described above, either reduce or eliminate the dryness of the hair.

Furthermore, the new composition helps to prevent split ends of the hair.

Various embodiments of the above composition and method may be implemented.

It will be recognized that the foregoing is but one example of a composition and method within the scope of the present invention and that various modifications will occur to those skilled in the art upon reading the disclosure set forth hereinbefore.

What is claimed is:

1. A composition for hair treatment comprising a mixture of gallnuts and copper oxide, each in powder form, in the ratio of about 75 gram copper oxide for each 1 kg of gallnuts, and a liquid solvent.

2. The composition for hair treatment according to claim 1, further including one or more of the following components:
cooking or olive oil and/or clove and/or almond shells and/or pomegranate shells and/or common salt (crystalline) and/or soap or wash paste and/or banana shells.

3. The composition for hair treatment according to claim 1, wherein the liquid solvent comprises vinegar and/or arrack in the ratio of about 8 teaspoons to 1 kg composition, and water.

4. The composition for hair treatment according to claim 1, wherein the liquid solvent comprises an alcoholic liquid, containing about 20% to 50% alcohol in water.

5. The composition for hair treatment according to claim 1, further inclding one or more of the following ingredients:
a. 5 grams of dry pomegranate peels.
b. 3 grams of dry banana peels.
c. 1.5 grams of dry artichoke leaves.
d. 2 grams of dry almond shells.
e. 5 grams of olive pits.

6. A method for preparing a composition for hair treatment, comprising the following steps:
A) Taking 1 kg. gall-nuts and dividing them up into 2 equal parts, a first part and a second part. Roasting with salt the first part for about 1 to 4 hours. Frying in oil the second part for about 15 minutes [11];
B) Mixing 150 grams of cloves with 75 grams of copper oxide, and grinding it up into powder. It is advisable to heat the cloves a little, for about 1 minute, before grinding them up [12];
C) Grinding up everything into powder and mixing [13];
D) Preparing a liquid solvent [14];
E) Mixing the liquid which has been prepared in Step D, with the powder which has been prepared in Step C. Mixing the above components initiates a chemical reaction, to create a compound with the stated properties [15].

7. The method according to claim 6, further including one or more of the following components: cooking or olive oil and/or clove and/or almond shells and/or pomegranate shells and/or common salt (crystalline) and/or soap or wash paste and/or banana shells.

8. The method according to claim 6, wherein the liquid solvent comprises vinegar and/or arrack in the ratio of about 8 teaspoons to 1 kg composition, and water.

9. The method according to claim 6, wherein the liquid solvent comprises an alcoholic liquid, containing about 20% to 50% alcohol in water.

10. The method according to claim 6, further including one of the following components or a combination thereof:
a. 5 grams of dry pomegranate peels.
b. 3 grams of dry banana peels.
c. 1.5 grams of dry artichoke leaves.
d. 2 grams of dry almond shells.
e. 5 grams of olive pits.

11. A method for preparing a composition for hair treatment, comprising the following steps:
A) Dividing a quantity of gallnuts into two halves, 0.5 kg each [31];
B) Frying half the gallnuts for about an hour, until the gallnuts “explode”, that is the shells open [32];
C) Frying the other half of the gallnuts in deep oil for about 20 to 30 minutes, until the gallnuts become soft [33];
D) Frying a quantity of copper oxide until blackening in a frying pan, for about 10 to 15 minutes, while mixing continuously [34];
E) Grinding separately each one of the above resulting components [35];
F) Mixing well all the components together [36];
G) Mixing the resulting powder or paste with water, in the ratio of 1 kg powder with 600 gram water [37];
H) Heating until boiling temperature and keeping it boiling for about 4 hours [38].

12. The method according to claim 11, wherein step (G) comprises in mixing about 10 cups of the composition with about 5 to 6 cups of water [37].

13. The method according to claim 11, further including the step of adding vinegar and/or arrack in the ratio of about 8 teaspoons to 1 kg composition.

14. The method according to claim 11, further including an alcoholic liquid, containing about 20% to 50% alcohol, to replace the water.

15. The method according to claim 11, further including one or more of the following components: cooking or olive oil and/or clove and/or almond shells and/or pomegranate shells and/or common salt (crystalline) and/or soap or wash paste and/or banana shells.

16. The method according to claim 11, further including one of the following components or a combination thereof:
a. 5 grams of dry pomegranate peels.
b. 3 grams of dry banana peels.
c. 1.5 grams of dry artichoke leaves.
d. 2 grams of dry almond shells.
e. 5 grams of olive pits.
